SEQUENCE LISTING (3 sequences)

SEQ ID NO: 1 - DNA, 20 bp, synthetic construct
  misc_feature 1..20: note = HKU-ORF1b-nsp14F
  source 1..20: mol_type = other DNA
tggggytttacrggtaacct

SEQ ID NO: 2 - DNA, 21 bp, synthetic construct
  misc_feature 1..21: note = HKU- ORF1b-nsp14R
  source 1..21: mol_type = other DNA
aacrcgcttaacaaagcactc

SEQ ID NO: 3 - DNA, 24 bp, synthetic construct
  misc_feature 1..24: note = HKU-ORF1b-nsp141P
  source 1..24: mol_type = other DNA
tagttgtgatgcwatcatgactag

(19) United States
(12) Patent Application Publication
(10) Pub. No.: US 2025/0152545 A1
(43) Pub. Date: May 15, 2025

(54) NOVEL ANTIVIRAL COMPOSITIONS COMPRISING OLEIC ACID

(71) Applicant: SUBINTRO LIMITED, London (GB)

(72) Inventors: William Garth RAPEPORT, London (GB); Kazuhiro ITO, London (GB); Jagdeep SHUR, London (GB)

(21) Appl. No.: 18/722,843

(22) PCT Filed: Dec. 23, 2022
(86) PCT No.: PCT/GB2022/053380

§ 371 Date: Jun. 21, 2024

(30) Foreign Application Priority Data
Dec. 23, 2021 (EP) 21217653.1

(51) Int. Cl. A61K 31/201; A61K 9/00; A61K 9/08; A61K 31/245; A61K 31/351; A61K 31/5377; A61K 31/765; A61K 47/10; A61K 47/12; A61K 47/36; A61K 47/38; A61P 31/14

(52) U.S. Cl. CPC A61K 31/201; A61K 9/0043; A61K 9/08; A61K 31/245; A61K 31/351; A61K 31/5377; A61K 31/765; A61K 47/10; A61K 47/12; A61K 47/36; A61K 47/38; A61P 31/14

(57) ABSTRACT

The invention provides inter alia a liquid pharmaceutical formulation suitable for topical administration to the lung or nose comprising a surfactant component selected from the group consisting of oleic acid or a pharmaceutically acceptable salt thereof and mixtures of oleic acid or a pharmaceutically acceptable salt thereof with polyoxyethylene sorbitan fatty acid esters optionally together with an anti-viral agent for use in the treatment or prevention of viral infection or disease associated with viral infection as a medicament for administration topically to the lung or nose.

### FIELD

This invention relates to liquid pharmaceutical formulations suitable for topical administration to the lung or nose and related aspects including canisters containing said formulations and spray and nebuliser devices and metered dose inhalers. The invention also relates to liquid pharmaceutical formulations for use in the treatment or prevention of viral infection and disease associated with viral infection, such as infection with SARS-COV-2 or influenza and related methods of treatment.

### BACKGROUND OF THE INVENTION

Severe acute respiratory syndrome coronavirus-2 (SARS-COV-2) is an enveloped positive-sense single-stranded RNA virus and a member of the genus Betacoronavirus of the family Coronaviridae. SARS-COV-2 is the causative agent of the respiratory disease, COVID-19 (Coronavirus disease 2019). COVID-19 is characterized by various degrees of severity, ranging from a mild upper respiratory illness to severe interstitial pneumonia and acute respiratory distress syndrome (ARDS), a life-threatening lung injury that allows fluid to leak into the lung. Post-COVID19 syndrome is also an important aspect, involving lung fibrosis and secondary lethal fungus infection/invasion.

At present, seven coronavirus species are known to cause disease in humans. The 229E, OC43, NL63 and HKU1 human coronavirus (hCoVs) species cause mild disease of the upper and lower respiratory tract and are estimated to account for one third of ‘common cold’ cases (Ludwig and Zarbock, 2020). However, the high prevalence, wide distribution, genetic diversity and frequent cross-species infection of coronavirus species facilitates emergence of novel human pathogens. Accordingly, severe acute respiratory syndrome coronavirus (SARS-CoV-1), Middle East respiratory syndrome-related coronavirus (MERS-COV) and more recently and most significantly, SARS-COV-2 have caused pandemics associated with a high mortality rate.

The principal mode of infection with SARS-COV-2 is the consequence of viral replication in the lining epithelium of the upper and lower respiratory tract. Initial infection with the virus initially follows the inhalation of very fine respiratory droplets and aerosol particles, deposition of respiratory droplets and particles on exposed mucous membranes in the mouth, nose, or eye by direct splashes and sprays, and touching mucous membranes with hands that have been soiled either directly by virus-containing respiratory fluids or indirectly by touching surfaces with virus on them (CDC: https://www.cdc.gov/coronavirus/2019-ncov/science/science-briefs/sars-cov-2-transmission.html). Robust levels of viral replication are established in the upper respiratory tract the virus in the early phase of the disease followed by migration into the lungs with the development of viral pneumonia and systemic spread to other organs. Available evidence suggests that disease severity is linked to viral load in the respiratory tract and that intervention with anti-viral agents reduce disease severity as a consequence of the reduction in viral burden.

In general, enveloped respiratory viruses attach to host cell surface receptors and enter cells by endocytosis or direct fusion of the virus membrane and the host cell membrane. Infection of upper and lower respiratory tract epithelial cells by SARS-COV-2 is facilitated by binding of the viral spike protein to the host cell receptor angiotensin converting enzyme 2 (ACE2). Other receptors, such as AXL, CD147, CD209/CD209L, neuropilin, DPP4 were identified as potential virologically relevant co-receptors (Xie et al.; Cantuti-Castelvetri et al.). Subsequent activation of the spike protein by host proteases such as TMPRSS on the apical surface is necessary for processing viral spike protein to enable virion entry by membrane fusion into the cell.

Recent studies demonstrate that SARS-COV-2 can enter human lung epithelial cells as host cells via two distinct pathways. Firstly, the cell surface direct membrane fusion (the early pathway) following spike protein activation by transmembrane serine protease 2 (TMPRSS2) or alternative serine proteases. Secondly, the endocytic uptake (the late pathway) whereby cathepsin L activates the spike protein in endosomal-lysosomal compartments. Critically, when TMPRSS2 or alternative serine proteases are expressed the early entry pathway is preferred, whereas when these proteases are absent, the virus relies on the late pathway (Murgolo et al. 2021). In the late pathway, processing and viral release in the endosome results from decreased pH and proteolysis which is needed to disrupt the viral envelope and release the genetic material inside. PIKfyve is a phosphoinositide kinase which phosphorylates phosphatidylinositol-3-phosphate (PI(3)P), to produce PI(3,5)P₂. PIKfyve plays a critical role in the maturation of endosomal membranes thereby enabling viral membrane fusion and entry into the cytoplasm.

There have been various reports concerning the potential use of PIKfyve inhibitors including apilimod to treat SARS-COV-2 infection. WO2021/211738 discloses anti-infective compositions useful for reducing the likelihood of a pathogenic infection, or for reducing transmission of said pathogen, said anti-infective composition comprising a compound selected from a group including apilimod. WO2021/158635 discloses PIKfyve inhibitors, such as apilimod, and composition thereof for treating or preventing coronavirus infections such as SARS-Cov-2. WO2016/161176 discloses a method of treating viral infection comprising administering to an individual in need thereof a therapeutically effective amount of a compound of general Formula (I);

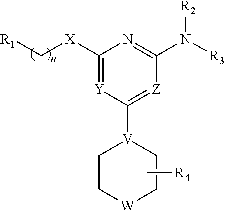

which encompasses apilimod.

In a number of cases, optimistic predictions concerning the usefulness of drugs to treat SARS-CoV-2 have proved unsuccessful in practice. For example, a number of early screening experiments to identify SARS-COV-2 anti-viral agents utilised the Vero E6 cell line. As this cell line is deficient in TMPRSS2 with high expression of ACE2, viral entry is dependent on the endocytic pathway and is thus an imperfect model for predicting infection of human epithelial cells. Furthermore, additional non-specific endocytic viral uptake mechanisms facilitate virion entry in Vero E6 cells. As such, many molecules, such as chloroquine and apilimod, that modulate the endosomal and lysosomal systems, have been identified as potent SARS-COV-2 drugs in Vero E6 cells although these observations may not translate to human lung epithelial cells (Hoffmann et al. 2020).

Further efforts to identify efficacious SARS-COV-2 anti-viral drugs has led to the discovery that camostat (as the mesylate salt) is capable of inhibiting SARS-COV-2 infection via blockade of TMPRSS2 and related proteases (Hoffmann et al. 2021). Furthermore, it has been established that remdesivir can be repurposed as a potent SARS-COV-2 drug (Pruijssers et al. 2020). In addition, the anti-coagulant nafamostat (as the mesylate salt) has been found to inhibit SARS-COV-2 infection of susceptible cells (Hoffmann et al. 2020). Alternatively, new SARS-COV-2 anti-virals may be designed. For example, molnupiravir, approved for use in the UK in November 2021, is an orally available SARS-COV-2 drug that inhibits the function of the viral RNA-dependent RNA polymerase (Kabinger et al. 2021). Similarly, nirmatrelvir has been developed as an inhibitor of the SARS-COV-2 3CL protease (Zhao et al. 2021). It has been found that combination of nirmatrelvir with the HIV protease inhibitor ritonavir slows metabolism of nirmatrelvir, thus prolonging activity (Zhao et al. 2021). Similarly, there are conflicting reports that lopinavir, an alternative HIV protease inhibitor, may have activity against SARS-COV-2, particularly when used in combination with ritonavir which increases the plasma concentration of lopinavir (Cattaneo et al. 2020; Ford et al. 2020).

Influenza viruses comprise four species, Influenza A-D viruses, each of which forms an individual genus, Alpha-, Beta-, Gamma- and Delta influenza virus respectively, within the Orthomyxoviridae family. Influenza viruses are enveloped negative-sense single-stranded RNA viruses with a segmented genome which cause mild to severe respiratory disease characterised by fever, sore throat, headache, coughing and fatigue. Influenza viruses utilise an endocytic route of entry. Binding of the viral HA protein to host sialic acid receptors triggers cellular entry via endosomal uptake. Subsequent acidification in the endosome activates cellular proteases such as Cathepsins and induces conformational changes in HA that enable endosome membrane fusion, thus releasing the virion into the cell. The reliance of influenza on the endocytic route for entry has led to speculation that PIKfyve inhibitors such as apilimod may be able to treat influenza virus infections. WO2021/211738 speculates that the anti-infective compositions of the invention, wherein said composition may comprise apilimod, may be used to treat influenza infections although no evidence of anti-influenza activity is presented.

Umifenovir, an influenza anti-viral drug that is widely used in Russia and China, is of significant clinical importance. Umifenovir binds to the viral HA protein and prevents the conformational changes in HA that enable membrane fusion from taking place (Kadam and Wilson, 2017). There are conflicting reports that umifenovir may also have activity against SARS-COV-2 (Huang et al. 2020).

SARS-COV-2 replication in human nasal and bronchial epithelium leads to a rapid loss of ciliated epithelium, characterized by impairment of muco-ciliary clearance, a decrease in epithelial integrity in the respiratory tract with disruption of epithelial tight junctions (Hao et al., 2020; Robinot et al., 2020. SARS-COV-2 is also reported to affect blood-gas barrier (BGB) or alveolar-capillary barrier is the primary tissue barner (Shirvaliloo, 2021).

Cytopathic infection requires a high viral load of 2.5 to 105 virions per cm2 of epithelium (Hao et al., 2020) Widespread damage to the ciliated epithelial layer of the upper respiratory tract precedes facilitate spread to the deeper lung parenchyma.

Thus, virus mediated damage to epithelial junction integrity causes loss of barrier to host defence where the consequence is inflammation, fluid leakage and loss of normal lung gas transfer as well as secondary infection with bacteria and fungus.

Conventional excipients used in pharmaceutical compositions for inhaled and intranasal delivery have traditionally been considered pharmaceutically inert substances serving to optimise the delivery of active pharmaceutical moieties for example, anti-allergic and anti-inflammatory agents. It has been reported in the literature that certain natural and synthetic surfactants may demonstrate anti-viral properties with reference to lipid enveloped viruses which are susceptible to the surfactant action.

Kohn et al. 1980 teaches that unsaturated fatty acids such as oleic acid exhibit virucidal In vitro activity against Sendai virus, Newcastle disease virus, Influenza A virus, Sindbis virus, West Nile virus and Herpes virus 1 but not against poliovirus, encephalomyocarditis virus or simian virus 40. The activity is proposed to be as a result of the incorporation into the viral lipid envelope thereby causing disruption of viral membrane integrity. However, this paper does not consider the effect of such molecules on host cell membranes, nor on human lung or nose epithelial cells, nor on coronaviruses or influenza virus.

Thormar et al. 1987 investigated the inactivation of viruses in fresh milk by fatty acids. The paper teaches that fatty acids such as oleic acid reduce virus titres following incubation with herpes simplex virus, VSV (vesicular stomatitis virus) and Visna virus although not poliovirus. It is reported that anti-viral fatty acids disrupt the integrity of the viral envelope, causing leakage and at higher concentrations a complete disintegration of the envelope and the virus particles, but they also caused disintegration of the plasma membranes of tissue culture cells resulting in cell lysis and death. However, this paper does not consider the effect of such molecules on human lung or nose epithelial cells or on coronaviruses or influenza virus.

Anderson et al. 1991 teaches that polysorbate 80 potentiates the anti-viral activity of the compound hypericin. However, it is disclosed that polysorbate 80 itself has no direct anti-viral activity when tested against vaccinia virus and herpes simplex virus strains.

Hilmarsson et al. 2007 teaches that oleic acid causes a significant reduction in the titre respiratory syncytial virus (RSV) following incubation in milk or fruit juice. However, this study teaches that the instability of long-chain unsaturated fatty acids such as oleic acid makes other microbicidal compounds such as lauryl alcohol, lauric acid, monolaurin and monocaprin more feasible active ingredients for topical formulations against RSV, parainfluenza or influenza infections. Furthermore, this paper does not consider the effect of oleic acid or similar molecules on lung or nose epithelial cells or on coronaviruses.

Chen et al. 2019 teaches that polysorbate 80, which is incapable of inducing inactivation of enveloped viruses, may be cleaved to oleic acid which is demonstrated to have anti-viral activity against pseudo-rabies virus and xenotropic murine leukaemia virus but not against porcine parvovirus. Accordingly, polysorbate 80 is suggested to be a viable replacement for Triton X-100 in manufacturing processes. However, this paper is limited to consideration of the effect of anti-viral surfactants in a biopharmaceutical manufacturing context and considers the effect of surfactants neither on host cell membranes nor on coronaviruses or influenza virus.

Alternative findings suggest that oleic acid may in fact lead to increased viral load:

Raini et al. 2021 teaches that supplementation of infected cells with oleic acids leads to increased Zika virus titres which may result from the induction of lipid droplet formation, since lipid droplets are a major platform for Zika virus replication.

Thus, there is great deal of conflicting and non-specific literature regarding the anti-viral effects of surfactants, such as polysorbate 80 and oleic acid.

There remains a need to develop pharmaceutical products for the treatment and prevention of viral infections, particularly those that affect epithelial cells of the lung or nose in humans.

### SUMMARY OF THE INVENTION

Commonly used excipients are generally regarded as pharmacologically inert. Surprisingly, the inventors have discovered new biological activities associated with certain surfactants which have previously been used as excipients.

The invention thus provides a liquid pharmaceutical formulation suitable for topical administration to the lung or nose having anti-viral properties comprising certain surfactant(s) and an anti-viral agent. The invention provides a liquid pharmaceutical formulation suitable for topical administration to the lung or nose comprising (i) a surfactant component selected from the group consisting of oleic acid or a pharmaceutically acceptable salt thereof and mixtures of oleic acid or a pharmaceutically acceptable salt thereof with polyoxyethylene sorbitan fatty acid esters and (ii) an anti-viral agent.

The invention also provides a liquid pharmaceutical formulation suitable for topical administration to the lung or nose comprising a surfactant component selected from the group consisting of oleic acid and mixtures of oleic acid with polyoxyethylene sorbitan fatty acid esters for use in the prevention of viral infection or disease associated with viral infection as a medicament for administration topically to the lung or nose.

### BRIEF DESCRIPTION OF THE FIGURES

FIG. 1 illustrates the experimental protocol described in Example 2.

FIG. 2 shows the effect of apically treating with apilimod (as mesylate), surfactants (0.15% w/w polysorbate 80 and 0.2% w/w oleic acid) and the combination thereof on SARS-COV-2 viral load in an apical wash from SARS-COV-2 infected air-liquid interface (ALI) cultured nasal epithelium. The effects of these treatments are compared to basolateral treatment with remdesivir.

FIG. 3 shows the effect of apically treating with apilimod (as mesylate), surfactants (0.15% w/w polysorbate 80 and 0.2% w/w oleic acid) and the combination thereof on SARS-COV-2 induced reduction of epithelial integrity determined by trans-epithelial electrical resistance (TEER). The effects of these treatments are compared to basolateral treatment with remdesivir.

FIG. 4 shows the effects of apically treated oseltamivir carboxylate (1 μM), surfactants (0.15% polysorbate 80 and 0.2% oleic acid) and the combination thereof on viral load in apical wash from H1N1-A/Switzerland/7717739/2013 (H1N1) infected air-liquid interface cultured nasal epithelium, compared with vehicle (water) treated infected control. H1N1 influenza virus particle was detected by RT-PCR, and genome copy numbers calculated from standard curve was shown. Oseltamivir carboxylate (10 μM) was also treated in basolateral chamber as an assay control.

FIG. 5 shows the effects of apically treated oseltamivir carboxylate (1 μM), surfactants (0.15% polysorbate 80 and 0.2% oleic acid) and the combination thereof on H1N1-influenza induced reduction of epithelial integrity compared with vehicle (water) treated infected control. Epithelial integrity was determined by TEER (trans-epithelial electrical resistance). Oseltamivir carboxylate (10 μM) was also treated in basolateral chamber as an assay control.

### DETAILED DESCRIPTION OF THE INVENTION

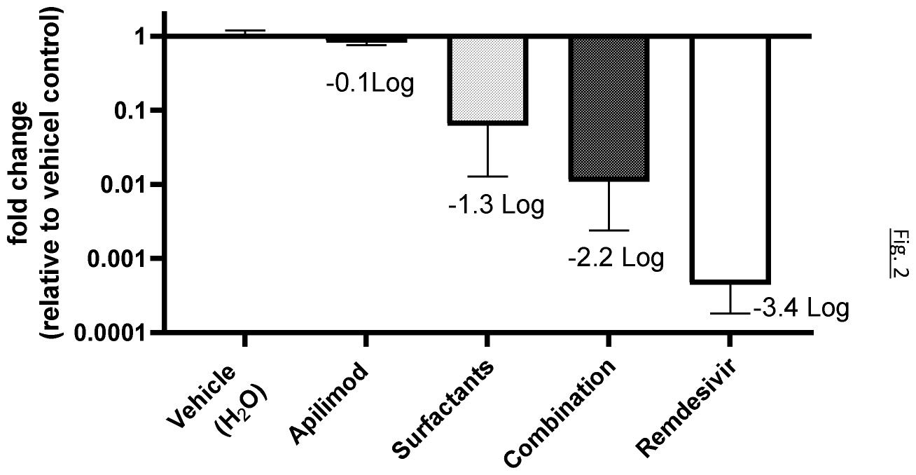

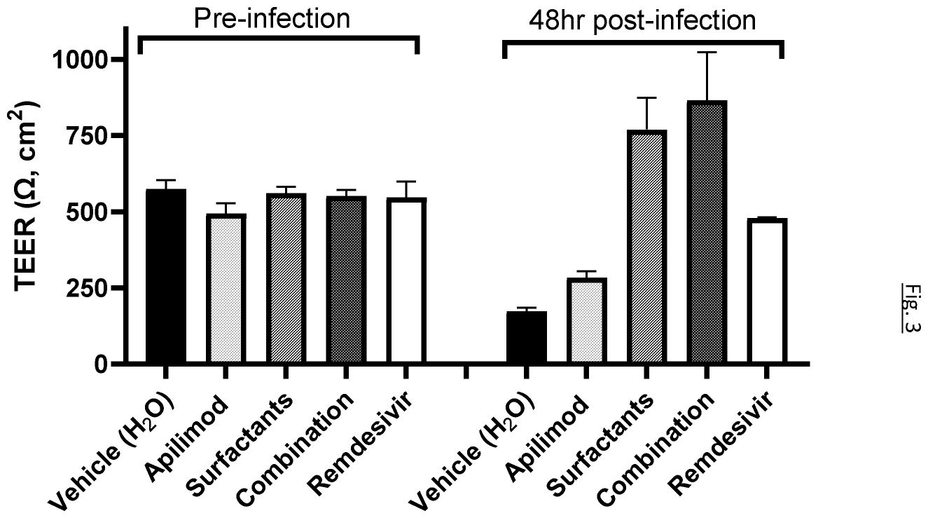

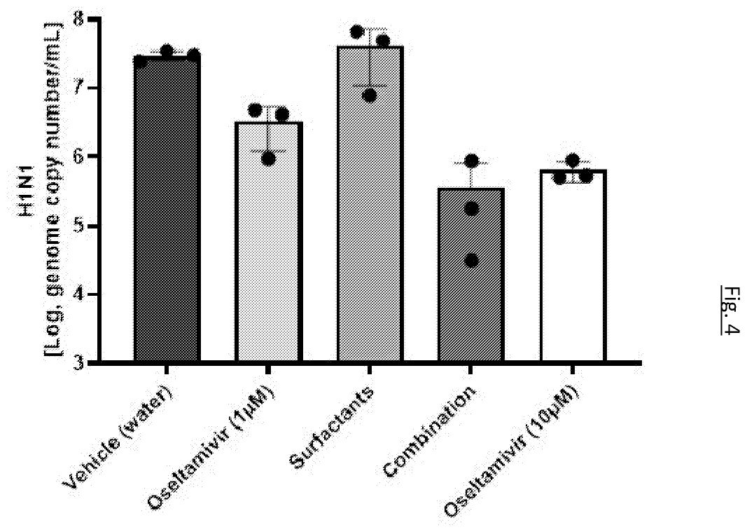

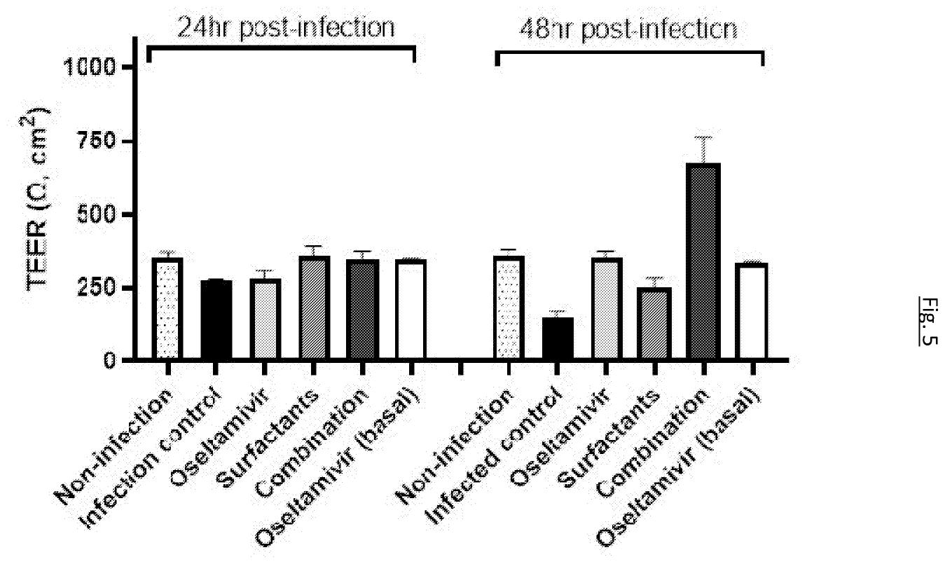

The present invention is based on discoveries made by testing the anti-viral activity of diverse anti-viral agents in combination with specific surfactants and the specific surfactants separately. The present invention is based on the discoveries that (i) apically administered mixture of oleic acid with polysorbate 80, particularly in combination with the anti-viral agent apilimod, has a powerful effect in reducing viral load in a model of infection of cultured nasal epithelium by SARS-COV-2 (see Example 2, FIG. 2) with evidence of synergy between the surfactant mixture and apilimod; (ii) apically administered mixture of oleic acid with polysorbate 80, optionally in combination with the anti-viral agent apilimod, has a powerful effect in improving barrier function in a model of infection of cultured nasal epithelium by SARS-COV-2 (see Example 2, FIG. 3); (iii) apically administered mixture of oleic acid with polysorbate 80 in combination with the anti-viral agent oseltamivir carboxylate has a powerful effect in reducing viral load in a model of infection of cultured nasal epithelium by influenza virus H1N1 (see Example 2, FIG. 4) with evidence of synergy between the surfactant mixture and oseltamivir carboxylate; and (iv) apically administered mixture of oleic acid with polysorbate 80, particularly in combination with the anti-viral agent oseltamivir carboxylate, has a powerful effect in improving barrier function in a model of infection of cultured nasal epithelium by influenza virus H1N1 (see Example 2, FIG. 5).

The liquid formulations of the invention comprise a surfactant component selected from the group consisting of oleic acid or a pharmaceutically acceptable salt thereof and mixtures of oleic acid or a pharmaceutically acceptable salt thereof with polyoxyethylene sorbitan fatty acid esters. Exemplary polyoxyethylene sorbitan fatty acid esters include polysorbate 80 (e.g. Tween 80) and polysorbate 20. For example, the surfactant component is oleic acid or a pharmaceutically acceptable salt thereof, especially oleic acid. More suitably the surfactant component is a mixture of oleic acid or a pharmaceutically acceptable salt thereof with polysorbate 20 or polysorbate 80, especially a mixture of oleic acid or a pharmaceutically acceptable salt thereof (such as oleic acid) with polysorbate 80.

Typically, the surfactant component may be present in the formulation at a concentration of 10-30000 ug/mL e.g. 100-20000 ug/mL e.g. 100-5000 ug/mL. For example, when the surfactant is oleic acid or a pharmaceutically acceptable salt thereof, the oleic acid may be present in the formulation at a concentration of 1-100 ug/mL. For example, when the surfactant component is a mixture of oleic acid or a pharmaceutically acceptable salt thereof with a polyoxyethylene sorbitan fatty acid ester such as polysorbate 20 or polysorbate 80, the oleic acid may be present in the formulation at a concentration of 10-30000 ug/mL e.g. 100-20000 ug/mL e.g. 100-5000 ug/mL and the polyoxyethylene sorbitan fatty acid ester such as polysorbate 20 or polysorbate 80 may be present in the formulation at a concentration of 10-20000 ug/mL e.g. 100-15000 ug/mL e.g. 100-5000 ug/mL. For example, the ratio of the amount of oleic acid or a pharmaceutically acceptable salt thereof to polyoxyethylene sorbitan fatty acid ester each measured in ug/mL is about 5:1 to 1:5. e.g. 2:1 to 1:2. The aforementioned amounts and ratios are based on the equivalent amount of free acid (oleic acid) when a salt form is used. Suitably oleic acid is used as oleic acid (i.e. not as a salt form).

Pharmaceutically acceptable salt forms of oleic acid that may be employed include sodium, potassium, ammonium salts and particularly the sodium salt. Most suitably oleic acid is used as the free acid.

The liquid pharmaceutical formulations of the invention will be suitable for topical administration to the lung or nose. The liquid pharmaceutical formulations of the invention may be administered by inhalation e.g. may be administered topically to the lung by oral inhalation or may be administered topically to the nose. It will be understood that formulations of the invention suitable for administration to the lung or nose when administered topically to the lung by oral inhalation or topically to the nose may thereby involve administration to the pharynx.

In certain embodiments the liquid pharmaceutical formulations of the invention comprise an anti-viral agent. Anti-viral agents include substances which have direct anti-viral activity e.g by inhibiting viral replication as well as those which have indirect anti-viral activity e.g. by impairing virus uptake for example by modulating the endosomal and lysosomal systems of target cells or by stimulating the host immune system to provoke an anti-viral response. In an embodiment, the anti-viral agent is apilimod (i.e. free base) or a pharmaceutically acceptable salt thereof such as the mesylate salt. In an embodiment, the anti-viral agent is camostat or a pharmaceutically acceptable salt thereof such as the mesylate salt. In an embodiment, the anti-viral agent is umifenovir. In an embodiment, the anti-viral agent is oseltamivir or a pharmaceutically acceptable salt thereof, particularly oseltamivir phosphate. In an embodiment, the anti-viral agent is ribavirin or a pharmaceutically acceptable salt thereof, particularly ribavirin. In an embodiment, the anti-viral agent is pirodavir or a pharmaceutically acceptable salt thereof, particularly pirodavir. In an embodiment, the anti-viral agent is remdesivir or a pharmaceutically acceptable salt thereof, particularly remdesivir. In an embodiment, the anti-viral agent is molnupiravir or a pharmaceutically acceptable salt thereof, particularly molnupiravir. In an embodiment, the anti-viral agent is a coronavirus 3CL protease inhibitor such as nirmatrelvir or a pharmaceutically acceptable salt thereof, particularly nirmatrelvir. In an embodiment, the anti-viral agent is lopinavir or a pharmaceutically acceptable salt thereof, particularly lopinavir. In an embodiment, the anti-viral agent is nirmatrelvir or a pharmaceutically acceptable salt thereof, particularly nirmatrelvir. In an embodiment, the anti-viral agent is nirmatrelvir or a pharmaceutically acceptable salt thereof, particularly nirmatrelvir, used in combination with ritonavir or a pharmaceutically acceptable salt thereof, particularly ritonavir. In an embodiment, the anti-viral agent is lopinavir or a pharmaceutically acceptable salt thereof, particularly lopinavir used in combination with ritonavir or a pharmaceutically acceptable salt thereof, particularly ritonavir. In an embodiment the anti-viral agent is baloxavir or a pharmaceutically acceptable salt thereof, particularly baloxavir marboxil. In an embodiment the anti-viral agent is ensitrelvir or a pharmaceutically acceptable thereof, particularly ensitrelvir fumarate. In an embodiment the anti-viral agent is favipiravir (T-705) or a pharmaceutically acceptable salt thereof, particularly favipiravir. In an embodiment the anti-viral agent is zanamivir or a pharmaceutically acceptable salt thereof, particularly zanamivir.

In an embodiment the anti-viral agent is oseltamivir or a pharmaceutically acceptable salt thereof, particularly oseltamivir phosphate. In an embodiment the anti-viral agent is oseltamivir carboxylate or a pharmaceutically acceptable salt. In an embodiment the anti-viral agent is laninamivir or a pharmaceutically acceptable salt thereof, particularly laninamivir. In an embodiment the anti-viral agent is laninamivir octanoate. In an embodiment the anti-viral agent is rupintrivir or a pharmaceutically acceptable salt thereof, particularly rupintrivir.

When the liquid pharmaceutical formulations of the invention are for the treatment or prevention of SARS COV-2 infection or COVID-19, suitably the anti-viral agent is selected from apilimod, camostat, nafamostat, umifenovir, remdesivir, molnupiravir, nirmatrelvir, lopinavir and pharmaceutically acceptable salts of any one thereof. Alternatively, suitably the anti-viral agent is selected from ensitrelvir, favipiravir, rupintrivir and pharmaceutically acceptable salts of any one thereof.

When the liquid pharmaceutical formulations of the invention are for the treatment or prevention of influenza virus infection or influenza, suitably the anti-viral agent is selected from camostat, nafamostat, umifenovir and pharmaceutically acceptable salts thereof. Alternatively, suitably the anti-viral agent is selected from baloxavir, favipiravir, oseltamivir, zanamivir, laninamivir, laninamivir octanoate and pharmaceutically acceptable salts thereof, particularly oseltamivir or oseltamivir carboxylate and pharmaceutically acceptable salts thereof.

When the liquid pharmaceutical formulations of the invention are for the treatment or prevention of respiratory syncytial virus (RSV) infection or disease associated with RSV, suitably the anti-viral agent is selected from ribavirin and pharmaceutically acceptable salts thereof.

When the liquid pharmaceutical formulations of the invention are for the treatment or prevention of human rhinovirus (HRV) infection or disease associated with HRV, suitably the anti-viral agent is selected from pirodavir, rupintrivir and pharmaceutically acceptable salts thereof.

When the liquid pharmaceutical formulations of the invention are for the treatment or prevention of coronavirus infection e.g. seasonal coronavirus infection e.g. 229E infection and the disease associated with viral infection is the disease associated with coronavirus infection e.g. seasonal coronavirus infection e.g. 229E infection, suitably the anti-viral agent is selected from apilimod, camostat, ensitrelvir, favipiravir, nafamostat, umifenovir, remdesivir, rupintrivir, molnupiravir, nirmatrelvir, lopinavir and pharmaceutically acceptable salts of any one thereof.

The details of the synthesis of the relevant anti-viral agents and ritonavir can be gleaned from the following documents: apilimod: WO2003/047516, camostat: Senokuchi et al., nafamostat: EP0048433B1, umifenovir: Chai et al. remdesivir: WO2017/184668, molnupiravir: US2020/0276219, nirmatrelvir: Owen et al., lopinavir: U.S. Pat. No. 5,914,332, ritonavir: U.S. Pat. No. 5,541,206, oseltamivir: U.S. Pat. No. 5,763,483, pirodavir: U.S. Pat. No. 5,231,184, ribavirin: USRE29835, baloxavir: JPWO2016/175224, ensitrelvir: Unoh et al., favipiravir: U.S. Pat. No. 6,800,629, rupintrivir: Dragovich et al., and zanamivir: U.S. Pat. No. 5,360,817, the contents of each of which document is incorporated by reference in its entirety. Nirmatrelvir is also known as PF07321332.

Pharmaceutically acceptable salts of basic compounds that may be used include acid addition salts such as hydrochloride, hydrobromide acetate, succinate and mesylate salts.

Typically, the anti-viral agent may be present in the formulation at a concentration of 0.01-2000 ug/mL e.g. 0.01-200 ug/mL. For example, when the anti-viral agent is apilimod (i.e. free base) or a pharmaceutically acceptable salt thereof (e.g. mesylate), the apilimod may be present in the formulation at a concentration of 2-200 ug/mL. For example, when the anti-viral agent is camostat (i.e. free base) or a pharmaceutically acceptable salt thereof (e.g. mesylate), the camostat may be present in the formulation at a concentration of 1 to 100 ug/mL. For example, when the anti-viral agent is nafamostat (i.e. free base) or a pharmaceutically acceptable salt thereof (e.g. mesylate), the nafamostat may be present in the formulation at a concentration of 0.1 to 100 ug/mL. For example, when the anti-viral agent is umifenovir or a pharmaceutically salt thereof, the umifenovir may be present in the formulation at a concentration of 10 to 10000 ug/mL. For example, when the anti-viral agent is remdesivir or a pharmaceutically salt thereof, the remdesivir may be present in the formulation at a concentration of 0.01-100 ug/mL. For example, when the anti-viral agent is molnupiravir, or a pharmaceutically acceptable salt thereof, the molnupiravir may be present in the formulation at a concentration of 0.01-10 ug/mL. For example, when the anti-viral agent is nirmatrelvir, or a pharmaceutically acceptable salt thereof, the nirmatrelvir may be present in the formulation at a concentration of 0.01-10 ug/mL. For example, when the anti-viral agent is lopinavir, or a pharmaceutically acceptable salt thereof, the lopinavir may be present in the formulation at a concentration of 1-1000 ug/mL. For example, when the anti-viral agent is oseltamivir, or a pharmaceutically acceptable salt thereof, the oseltamivir may be present in the formulation at a concentration of 0.01-100 ug/mL. For example, when the anti-viral agent is oseltamivir carboxylate, or a pharmaceutically acceptable salt thereof, the oseltamivir carboxylate may be present in the formulation at a concentration of 0.01-100 ug/mL. For example, when the anti-viral agent is pirodavir, or a pharmaceutically acceptable salt thereof, the pirodavir may be present in the formulation at a concentration of 0.1-100 ug/mL. For example, when the anti-viral agent is ribavirin, or a pharmaceutically acceptable salt thereof, the ribavirin may be present in the formulation at a concentration of 1-1000 ug/mL. For example, when the anti-viral agent is baloxavir, or a pharmaceutically acceptable salt thereof, the baloxavir may be present in the formulation at a concentration of 0.01-10 ug/mL. For example, when the anti-viral agent is ensitrelvir, or a pharmaceutically acceptable salt thereof, the ensitrelvir may be present in the formulation at a concentration of 0.01-100 ug/mL. For example, when the anti-viral agent is favipiravir, or a pharmaceutically acceptable salt thereof, the favipiravir may be present in the formulation at a concentration of 1-1000 ug/mL. For example, when the anti-viral agent is zanamivir, or a pharmaceutically acceptable salt thereof, the zanamivir may be present in the formulation at a concentration of 0.01-100 ug/mL. For example, when the anti-viral agent is laninamivir, or a pharmaceutically acceptable salt thereof, the laninamivir may be present in the formulation at a concentration of 0.01-100 ug/mL. For example, when the anti-viral agent is laninamivir octanoate, or a pharmaceutically acceptable salt thereof, the laninamivir octanoate may be present in the formulation at a concentration of 0.01-100 ug/mL. For example, when the anti-viral agent is rupintrivir, or a pharmaceutically acceptable salt thereof, the rupintrivir may be present in the formulation at a concentration of 0.01-100 ug/mL.

Ritonavir, or a pharmaceutically acceptable salt thereof, is a potent inhibitor of intestinal and hepatic cytochrome p450 3A4 and may be included in the liquid pharmaceutical formulation to enhance the effect of another anti-viral agent such as nirmatrelvir or lopinavir (Choy et al.). For example, when ritonavir, or a pharmaceutically acceptable salt thereof is to be included, the ritonavir may be present in the formulation at a concentration of 0.005-5 ug/mL.

For example, when the anti-viral agent is nirmatrelvir or a pharmaceutically acceptable salt thereof used in combination with ritonavir or a pharmaceutically acceptable salt thereof, the nirmatrelvir may be present in the formulation at a concentration of 0.01-10 ug/mL and the ritonavir may be present in the formulation at a concentration of 0.005-5 ug/mL. For example, when the anti-viral agent is lopinavir or a pharmaceutically acceptable salt thereof used in combination with ritonavir or a pharmaceutically acceptable salt thereof, the lopinavir may be present in the formulation at a concentration of 1-1000 ug/mL and the ritonavir may be present in the formulation at a concentration of 0.25-250 ug/mL. The aforementioned amounts are based on the equivalent amount of free form (e.g. base/acid) of the anti-viral agent when a salt form is used.

The liquid pharmaceutical formulation may be a solution formulation in which the anti-viral agent is dissolved in the formulation. In the case of solution formulations, a soluble form of the anti-viral agent will be selected. For example, the mesylate salt of apilimod is soluble in water.

Alternatively, an aqueous pharmaceutical formulation may be a suspension formulation comprising the anti-viral agent as a solid in finely divided form. When the solid is in finely divided form it will be of a size suitable for the intended route of delivery i.e. to the lung or nose.

In certain embodiments the liquid pharmaceutical formulation may be an aqueous formulation in which the anti-viral agent may be dissolved or suspended in the formulation. In the case that that the anti-viral agent is dissolved in the aqueous formulation (i.e. the formulation is a solution formulation), a soluble form of the anti-viral agent will be selected. For example, the mesylate salt of apilimod is soluble in water. The dissolution of the anti-viral agent may be aided by inclusion in the formulation of a solvent, such as polar organic solvent e.g. an alcohol, a polyol or a polyethyleneglycol (PEG) e.g. ethanol. In the case that the anti-viral agent is suspended in the aqueous formulation, a relatively insoluble form of the anti-viral agent will be selected. For example, the apilimod base is relatively insoluble in water. In such suspension formulations the anti-viral agent will be in solid and finely divided form of a size suitable for the intended route of delivery i.e. to the lung or nose. In the case of an aqueous formulation, the water used in the formulation will be sterilised water. Aqueous formulations may optionally include other ingredients such as preservatives, buffers and osmotic agents. Example preservatives include edetic acid and their alkali salts such as disodium EDTA (also referred to as “disodium edetate” or “the disodium salt of edetic acid”) and calcium EDTA (also referred to as “calcium edetate”), benzyl alcohol, methylparaben, propylparaben, butylparaben, chlorobutanol, phenylethyl alcohol, benzalkonium chloride, thimerosal, propylene glycol, sorbic acid, and benzoic acid derivatives. Example buffers include buffers based on weak organic acids such as citrate/citric acid buffers. Osmotic agents increase the osmolarity of the formulation therefore increasing patient comfort. Example osmotic agents include polyols such as sugar and sugar alcohols e.g. xylitol and glycerol (glycerine). Aqueous formulations suitable for topical administration to the nose may optionally contain wetting agents and thickening agents e.g. hyaluronic acid or a pharmaceutically acceptable salt thereof. Aqueous suspension formulations may optionally contain suspending agents to aid the maintenance of a suspension such as surfactants and cellulose derivatives such as microcrystalline cellulose/carboxymethylcellulose sodium (Avicel), carrageenans and hyaluronic acid or a pharmaceutically acceptable salt thereof. The surfactant component in the formulation may serve as or contribute to the function of a suspending agent.

The pH of the aqueous liquid pharmaceutical formulation can typically be in a wide range e.g. between 4 and 8.

In certain embodiments the aqueous liquid pharmaceutical formulation may be a micellar solution where the hydrophilic “head” region of the surfactant molecules are in contact with surrounding solvent, sequestering the hydrophobic single-tail regions in the micelle centre formed from the “tail” regions of the surfactant molecules.

In certain embodiments the liquid pharmaceutical formulation may be pressurised liquid formulation in which the anti-viral agent may be dissolved or more preferably suspended in the formulation. In the case of pressurised liquid suspension formulations, a relatively insoluble form of the anti-viral agent will be selected. For example, the mesylate salt of apilimod and its base form are relatively insoluble in pressurised liquids such as hydrofluoroalkanes. In the case of pressurised liquid solution formulations, the dissolution of the anti-viral agent may be aided by inclusion in the formulation of a solvent, such as a polar organic solvent e.g. ethanol. In suspension formulations, the anti-viral agent will be in a solid finely divided form. The surfactant component in the formulation may serve as or contribute to the function of a suspending agent to aid maintenance of the suspension. When the solid is in finely divided form it will be of a size suitable for the intended route of delivery i.e. to the lung or nose.

Typically, the particles of the solid in finely divided form in the above-mentioned formulations will have a mass median aerodynamic diameter (MMAD) in the range 1-10 μm Particles of an appropriate size may be produced by air-jet milling, spray drying, supercritical fluid extraction or nano-milling. The MMAD of particles of the solid may be determined using a next generation impactor (NGI) (Marple et al. 2021).

The pressurised liquid used in pressurised liquid formulations is suitably a volatile non-polar liquid such as a hydrofluoroalkane (HFA) or hydrofluoroolefin (HFO) such as HFA134a, HFA227, HFA152a, HFO1234ze or HFO1234zf or a mixture thereof, especially HFA134a, HFA 152a or HFO1234ze.

In some embodiments, non-pressurised liquid pharmaceutical formulations may be administered to the nose via a nasal spray device or nasal drop applicator. Nasal spray devices and nose drop applicators are known in the art and disclosed in U.S. Pat. Nos. 2,577,321 and 6,000,580 the contents of each of which are herein incorporated by reference in their entirety. As used herein, the term “nose drop applicator” refers to any dispenser suitable for administration of nose drops. The nasal spray device will typically administer a metered volume of liquid e.g. it may administer a volume of 50 to 200 μL especially 100 μL. The metered volume is suitably administered to each nostril (e.g. one or two times per nostril).

In some embodiments, non-pressurised liquid pharmaceutical formulations especially aqueous formulations such as aqueous solution formulations may be administered via a nebuliser device. Nebulisers produce aerosols for inhalation typically in a continuous manner as long as they are switched on or breath-actuated. Nebuliser devices can be hand-held and portable or for home or hospital use (i.e. non-portable). Established nebuliser products include Aeroneb® and Pari® devices and are disclosed in U.S. Pat. No. 9,364,618 the contents of which are herein incorporated by reference in their entirety. Nebuliser devices may for example be piezeoelectric nebuliser devices which are known to produce homogenous aerosols based on the high frequency vibration of a metal (typically a stainless steel) mesh or membrane containing small holes (typically micrometer size). The aerosol generated may be directed to the lung or lung and nose by inhalation by means of a suitable mouth or nose piece. When formulations of the invention are administered topically to the lung by oral inhalation or topically to the nose, the formulation may thereby be administered to the pharynx.

Pressurised formulations will generally be retained in a canister (e.g. an aluminium canister) closed with a valve (e.g. a metering valve) and fitted into an actuator provided with a mouthpiece.

Canisters generally comprise a container capable of withstanding the vapour pressure of the HFA propellant, such as plastic or plastics coated glass bottle or preferably a metal can, for example an aluminium can which may optionally be anodised, lacquer-coated and/or plastics coated, which container is closed with a metering valve. It may be preferred that canisters be coated with a fluorocarbon polymer as described in WO 96/32151, for example, a co-polymer of polyethersulphone (PES) and polytetrafluoroethylene (PTFE). Another polymer for coating that may be contemplated is FEP (fluorinated ethylene propylene). The metering valves are designed to deliver a metered amount of the formulation per actuation and incorporate a gasket to prevent leakage of propellant through the valve. The gasket may comprise any suitable elastomeric material such as for example low density polyethylene, chlorobutyl, black and white butadiene-acrylonitrile rubbers, butyl rubber and neoprene. Thermoplastic elastomer valves as described in WO92/11190 and valves containing EPDM rubber as described in WO95/102651 are especially suitable. Suitable valves are commercially available from manufacturers well known in the aerosol industry, for example, from Valois, France (eg. DF10, DF30, DF60), Bespak pic, UK (eg. BK300, BK356, BK357) and 3M-Neotechnic Ltd, UK (eg. Spraymiser™). The DF31 valve of Valois, France is also suitable.

Valve seals, especially the gasket seal, will preferably be manufactured of a material which is inert to and resists extraction into the contents of the formulation, especially when the contents include ethanol. Valve materials, especially the material of manufacture of the metering chamber, will preferably be manufactured of a material which is inert to and resists distortion by contents of the formulation, especially when the contents include ethanol. Particularly suitable materials for use in manufacture of the metering chamber include polyesters eg. polybutyleneterephthalate (PBT) and acetals, especially PBT. Materials of manufacture of the metering chamber and/or the valve stem may desirably be fluorinated, partially fluorinated or impregnated with fluorine containing substances in order to resist drug deposition. The valve chamber will be of a size appropriate for the dose to be dispensed and the concentration of the liquid pharmaceutical formulation e.g. 25-100 uL e.g. 25 L or 100 uL.

Conventional bulk manufacturing methods and machinery well known to those skilled in the art of pharmaceutical aerosol manufacture may be employed for the preparation of large scale batches for the commercial production of filled canisters. Thus, for example, in one bulk manufacturing method a metering valve is crimped onto an aluminium can to form an empty canister. The formulation containing the medicament, propellant and any other formulation ingredients is pressure filled through the charge vessel into a manufacturing vessel. Typically, in batches prepared for pharmaceutical use, each filled canister is check-weighed, coded with a batch number and packed into a tray for storage before release testing. In an alternative process, an aliquot of the liquified formulation is added to an open canister under conditions which are sufficiently cold that the formulation does not vaporise, and then a metering valve crimped onto the canister. Typically, in batches prepared for pharmaceutical use, each filled canister is check-weighed, coded with a batch number and packed into a tray for storage before release testing.

Each filled canister is conveniently fitted into a suitable channelling device prior to use to form a metered dose inhaler for administration of the medicament into the lungs or nasal cavity of a patient. Suitable channelling devices comprise, for example a valve actuator and a cylindrical or cone-like passage through which medicament may be delivered from the filled canister via the metering valve to the nose or mouth of a patient e.g. a mouthpiece actuator. Metered dose inhalers are designed to deliver a fixed unit dosage of medicament per actuation or ‘puff’, for example in the range of 10 to 5000 ug medicament per puff.

In a typical arrangement the valve stem is seated in a nozzle block which has an orifice leading to an expansion chamber. The expansion chamber has an exit orifice which extends into the mouthpiece. Actuator (exit) orifice diameters in the range 0.1-0.45 mm are generally suitable eg. 0.15, 0.22, 0.25, 0.30, 0.33 or 0.42 mm. The dimensions of the orifice should not be so small that blockage of the jet occurs.

Actuator jet lengths are typically in the range 0.30-1.7 mm e.g. 0.30, 0.65 or 1.50 mm. for buccal administration (i.e. oral administration).

The precise shape and dimensions of the actuator will be adapted for topical administration to the lung or nose as appropriate.

An aspect of the invention is a liquid pharmaceutical formulation as described herein for use as a medicament for administration topically to the lung or nose and in particular in the treatment or prevention of infection by a virus or disease associated with infection with such a virus. In one embodiment, the liquid pharmaceutical formulation is for use in the treatment of viral infection or disease associated with viral infection. In another embodiment, the liquid pharmaceutical formulation is for use in the prevention of viral infection or disease associated with viral infection.

A further aspect of the invention is a method of treating or preventing viral infection or disease associated with infection by a virus or disease associated with infection with such a virus which comprises administering to a subject in need thereof topically to the lung or nose a therapeutically or prophylactically effective amount of a pharmaceutical formulation as described herein. In one embodiment, the method is a method of treating viral infection or disease associated with viral infection. In another embodiment, the method is a method of preventing viral infection or disease associated with viral infection.

A further aspect of the invention is use of a liquid pharmaceutical formulation as described herein for the manufacture of a medicament for administration topically to the lung or nose and in particular for the treatment or prevention of infection by a virus or disease associated with infection with such a virus.

An aspect of the invention is a liquid pharmaceutical formulation suitable for topical administration to the lung or nose comprising a surfactant component selected from the group consisting of oleic acid or a pharmaceutically acceptable salt thereof and mixtures of oleic acid or a pharmaceutically acceptable salt thereof with polyoxyethylene sorbitan fatty acid esters for use in the treatment or prevention of viral infection or disease associated with viral infection as a medicament for administration topically to the lung (e.g. by oral inhalation) or nose. In one embodiment, the liquid pharmaceutical formulation is for use in the treatment of viral infection or disease associated with viral infection. In another embodiment, the liquid pharmaceutical formulation is for use in the prevention of viral infection or disease associated with viral infection.

A further aspect of the invention is a method of treating or preventing viral infection or disease associated with viral infection which comprises administering to a subject in need thereof topically to the lung (e.g. by oral administration) or nose a therapeutically or prophylactically effective amount of a pharmaceutical formulation comprising a surfactant component selected from the group consisting of oleic acid or a pharmaceutically acceptable salt thereof and mixtures of oleic acid or a pharmaceutically acceptable salt thereof with polyoxyethylene sorbitan fatty acid esters. In one embodiment, the method is a method of treating viral infection or disease associated with viral infection. In another embodiment, the method is a method of preventing viral infection or disease associated with viral infection.

A further aspect of the invention is use of a liquid pharmaceutical formulation suitable for topical administration to the lung or nose comprising a surfactant component selected from the group consisting of oleic acid or a pharmaceutically acceptable salt thereof and mixtures of oleic acid or a pharmaceutically acceptable salt thereof with polyoxyethylene sorbitan fatty acid esters for the manufacture of a medicament for use in the prevention of viral infection or disease associated with viral infection for administration topically to the lung (e.g. by oral inhalation) or nose.

A further aspect of the invention is a liquid pharmaceutical formulation suitable for topical administration to the lung (e.g. by oral inhalation) or nose comprising a surfactant component selected from the group consisting of oleic acid or a pharmaceutically acceptable salt thereof and mixtures of oleic acid or a pharmaceutically acceptable salt thereof with polyoxyethylene sorbitan fatty acid esters for use in improving epithelial barrier function (particularly the barrier function of lung or nose epithelia) in vivo.

A further aspect of the invention is a liquid pharmaceutical formulation suitable for topical administration to the lung (e.g. by oral inhalation) or nose comprising (i) a surfactant component selected from the group consisting of oleic acid or a pharmaceutically acceptable salt thereof and mixtures of oleic acid or a pharmaceutically acceptable salt thereof with polyoxyethylene sorbitan fatty acid esters and (ii) an anti-viral agent for use in improving epithelial barrier function (particularly the barrier function of lung or nose epithelia) in vivo.

A further aspect of the invention is a method of improving epithelial barrier function (particularly the barrier function of lung or nose epithelia) in a subject which comprises administering topically to epithelia of the lung (e.g. by oral inhalation) or nose of said subject a liquid pharmaceutical formulation comprising a surfactant component selected from the group consisting of oleic acid or a pharmaceutically acceptable salt thereof and mixtures of oleic acid or a pharmaceutically acceptable salt thereof with polyoxyethylene sorbitan fatty acid esters.

A further aspect of the invention is a method of improving epithelial barrier function (particularly the barrier function of lung or nose epithelia) in a subject which comprises administering topically to epithelia of the lung (e.g. by oral inhalation) or nose of said subject a liquid pharmaceutical formulation comprising (i) surfactant component selected from the group consisting of oleic acid or a pharmaceutically acceptable salt thereof and mixtures of oleic acid or a pharmaceutically acceptable salt thereof with polyoxyethylene sorbitan fatty acid esters and (ii) an anti-viral agent.

For example, in respect of the above aspects, the viral infection is a coronavirus infection. For example, in respect of the above aspects, the viral infection is SARS-COV-2 infection and the disease associated with viral infection is COVID-19. For example, in respect of the above aspects, the viral infection is seasonal coronavirus, for example 229E and the disease associated with viral infection is the disease associated with seasonal coronavirus, for example 229E, infection. For example, in respect of the above aspects, the viral infection is influenza virus infection and the disease associated with viral infection is influenza. For example, in respect of the above aspects, the viral infection is respiratory syncytial virus (RSV) and the disease associated with viral infection is the disease associated with RSV infection. For example, in respect of the above aspects, the viral infection is human rhinovirus (HRV) and the disease associated with viral infection is the disease associated with HRV infection. As used herein, “influenza virus” includes influenza A virus, influenza B virus, influenza C virus and influenza D virus, for example influenza A virus or influenza B virus.

In methods of prevention of viral infection, or disease associated with viral infection, the liquid pharmaceutical formulation will suitably be administered to the subject in need thereof approximately 1-3 days prior to potential exposure to viral infection e.g. daily up to 1-4 times daily.

In methods of treatment of viral infection, or disease associated with viral infection, the liquid pharmaceutical formulation will suitably be first administered to the subject in need thereof within 72 hours, suitably within 48 hours, of exposure to viral infection. Treatment administrations will typically be made for 3-10 days e.g for 5-10 days.

In methods described herein, the liquid pharmaceutical formulation will be administered with a frequency of 1-4 times daily.

A suitable dose of the liquid pharmaceutical formulation per administration for use in methods as described herein is a therapeutically or prophylactically effective dose which can be determined by the skilled person.

Suitably, treatments or preventions as described herein are for treatment or prevention of virus infection or disease in mammals, especially humans.

An alternative aspect of the invention relates to a liquid pharmaceutical formulation suitable for topical administration to the lung or nose comprising a surfactant component selected from the group consisting of oleic acid or a pharmaceutically acceptable salt thereof and mixtures of oleic acid or a pharmaceutically acceptable salt thereof with polyoxyethylene sorbitan fatty acid esters. Suitably said formulation is an aqueous formulation. Suitable amounts and concentrations of surfactants are discussed above. Such a formulation may be for use in treating or preventing viral infection or disease associated with viral infection. The invention also provides a method of treatment or prevention of said infection/disease comprising administering to a subject in need thereof a therapeutically or prophylactically effective amount of said pharmaceutical formulation. The invention also provides use of such a formulation as described herein for the manufacture of a medicament for administration topically to the lung or nose and in particular for the treatment or prevention of infection by a virus or disease associated with infection with such a virus. Example infections/diseases are as discussed above and include SARS-COV-2/COVID-19 and influenza virus infection/influenza. Formulations may be administered topically to lung e.g. by oral inhalation or topically to the nose.

As used herein, “subject” is suitably a human subject.

Treatment of virus infection after exposure may lead to prevention of disease hence “treatment” of virus infection includes post exposure prevention of disease associated with virus infection.

In the formulations of the invention, the surfactant(s) and anti-viral agent when co-formulated have favourable pharmaceutical and biological properties. However, the components of the formulation may alternatively be co-administered in separate formulations. Thus the invention also provides a liquid pharmaceutical formulation suitable for topical administration to the lung or nose comprising a surfactant component selected from the group consisting of oleic acid or a pharmaceutically acceptable salt thereof and mixtures of oleic acid or a pharmaceutically acceptable salt thereof with polyoxyethylene sorbitan fatty acid esters for use in combination with a liquid pharmaceutical formulation suitable for topical administration to the lung or nose comprising an anti-viral agent whereby the formulation comprising the surfactant component and the formulation comprising the anti-viral agent are co-administered. The invention also provides a method of treating or preventing viral infection or disease associated with infection by a virus or disease associated with infection with such a virus which comprises co-administering (i) a liquid pharmaceutical formulation suitable for topical administration to the lung or nose comprising a surfactant component selected from the group consisting of oleic acid or a pharmaceutically acceptable salt thereof and mixtures of oleic acid or a pharmaceutically acceptable salt thereof with polyoxyethylene sorbitan fatty acid esters and (ii) a liquid pharmaceutical formulation suitable for topical administration to the lung or nose comprising an anti-viral agent. Formulations may be administered topically to the lung e.g. by oral inhalation or topically to the nose. By “co-administered” in the context is meant that the two formulations are administered at essentially the same time e.g. at the same time or within a few seconds (e.g. 1-3 seconds) of each other.

### EXAMPLES

Abbreviations used herein are defined below (Table 1). Any abbreviations not defined are intended to convey their generally accepted meaning.

TABLE 1
Abbreviations

| | |
|---|---|
| ALI | air liquid interface |
| Ct | Cycle threshold |
| hr | hour(s) |
| MOI | multiplicity of infection |
| ORF | Open reading frame |
| PBS | phosphate buffered saline |
| PCR | polymerase chain reaction |
| SARS-COV-2 | severe acute respiratory syndrome coronavirus 2 |
| TEER | trans-epithelial electrical resistance |

### Materials

All starting materials and solvents were obtained from commercial sources.

### Example 1A: Example Liquid Pharmaceutical Formulation of Apilimod for Administration to the Nose Via a Nasal Spray Device

The following aqueous suspension formulation may be prepared:

| Formulation | | State in | Concentration in | Amount |
|---|---|---|---|---|
| Component | Function | Formulation | Drug Product | Per Spray |
| Apilimod | Active | Suspension | 2.00% w/w | 2000 μg |
| | Ingredient | | | |
| Oleic Acid | Active | Solution | 2.00% w/w | 2000 μg |
| | ingredient | | | |
| Polysorbate 80 | Active | Solution | 1.50% w/w | 1500 μg |
| | ingredient | | | |
| Microcrystalline cellulose/ | Suspending | Suspension | 2.00% w/w | 2000 μg |
| carboxymethylcellulose | Agent | | | |
| Sodium (Avicel RC591) | | | | |
| Hyaluronic acid | Suspending/ | Suspension | 1.00% w/w | 1000 μg |
| | wetting/ | | | |
| | thickening | | | |
| | agent | | | |
| Glycerin | Osmotic | Solution | 2.1% w/w | 2100 μg |
| | Agent | | | |
| Citric Acid, Monohydrate | Buffer | Solution | 0.20% w/w | 200 μg |
| Sodium Citrate, Dihydrate | Buffer | Solution | 0.28% w/w | 280 μg |
| Purified Water | Diluent | | q.s. | 88.92 mg |

The formulation may be administered to the nose via a nasal spray device (e.g. spray volume 100 μL, 1-2 sprays per nostril) for the prevention or treatment of virus infection e.g. by SARS-CoV-2.

### Example 1B: Example Liquid Pharmaceutical Formulation of Zanamivir for Administration to the Nose Via a Nasal Spray Device

The following aqueous suspension formulation may be prepared:

| Formulation | | State in | Concentration in | Amount |
|---|---|---|---|---|
| Component | Function | Formulation | Drug Product | Per Spray |
| Zanamivir | Active | Solution | 5.00% w/w | 5000 μg |
| | Ingredient | | | |
| Oleic Acid | Active | Solution | 2.00% w/w | 2000 μg |
| | ingredient | | | |
| Polysorbate 80 | Active | Solution | 1.50% w/w | 1500 μg |
| | ingredient | | | |
| Microcrystalline cellulose/ | Suspending | Suspension | 2.00% w/w | 2000 μg |
| carboxymethylcellulose | Agent | | | |
| Sodium (Avicel RC591) | | | | |
| Hyaluronic acid | Suspending/ | Suspension | 1.00% w/w | 1000 μg |
| | wetting / | | | |
| | thickening | | | |
| | agent | | | |
| Glycerin | Osmotic | Solution | 2.1% w/w | 2100 μg |
| | Agent | | | |
| Citric Acid, Monohydrate | Buffer | Solution | 0.20% w/w | 200 μg |
| Sodium Citrate, Dihydrate | Buffer | Solution | 0.28% w/w | 280 μg |
| Purified Water | Diluent | | q.s. | 85.92 mg |

The formulation may be administered to the nose via a nasal spray device (e.g. spray volume 100 μL, 1-2 sprays per nostril) for the prevention or treatment of virus infection e.g. by influenza.

### Example 2: Assessment of Virus Load and Cell Integrity in Air-Liquid Interface (ALI) Cultured Human Epithelial Cells Infected with SARS-COV-2-Studies with Apilimod

### Experimental Methods

Primary human nasal, trachea, and bronchial epithelial cells can be cultured and differentiated at an air-liquid interface (ALI), forming a pseudostratified mucociliary airway epithelium that is composed of ciliated cells, goblet cells, club cells, and basal cells with an arrangement closely reflective of an in vivo cellular organization. This in vitro model of human airway epithelium (HAE) cultured at an ALI (HAE-ALI) closely recapitulates many important characteristics of respiratory virus-host cell interactions seen in the infected upper and lower airways in vivo and has been used to study many human respiratory viruses, including SARS-COV-2. Notably, differentiation at an ALI results in a drastic increase in expression and the polar presentation of the viral receptor ACE2 on the apical membrane as well as high level of expression of TMPRSS 2 Thus, HAE-ALI is an optimal cell culture model to study SARS-COV-2 infection in vitro.

ALI cultured pooled donors' human nasal epithelium (provided by Epithelix Sarl (Geneva, Switzerland)) were maintained in air-liquid interphase with MucilAir™ culture media in Costar Transwell inserts (Corning, NY, USA) according to the manufacturer's instructions and used for SARS-COV-2 infection (in VirNext, University of Lyon) as previously reported (Pizzorno et al., 2020, Cell Rep Med.1(4): 100059). On Day 0, SARS-COV-2 inoculum (strain BetaCoV/France/IDF0571/2020 (accession ID EPI_ISL_411218), 100 uL; diluted in MucilAir culture medium to give a at a multiplicity of infection final MOI of 0.1) was added to apical surface for 1 hr (37° C./5% CO2). Virus inoculum was removed, and inserts were washed with sterile PBS (with Ca2+/Mg2+).

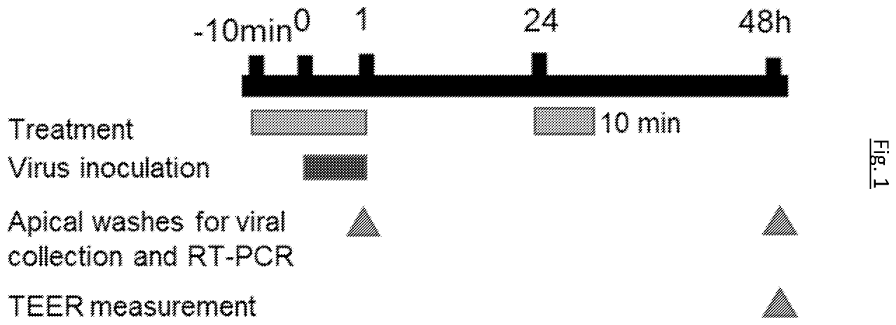

As illustrated in FIG. 1, ALI cultures were dosed apically with apilimod (as mesylate) dissolved in water (2 mg/ml, 50 uL), surfactants (0.15% w/w polysorbate 80 and 0.2% w/w oleic acid) or apilimod (as mesylate) and surfactants at 10 min prior to virus inoculation, and further 60 min together with virus inoculum on Day 0 (then being removed together with virus inoculum as indicated above), and also re-applied to the apical surface on Day 1 (24 hrs post inoculation) for 10 min before being removed. Vehicle treatments (water) were performed on the corresponding apical surfaces to ensure each well received the same number of manipulations. In addition, remdesivir (5 μM) was added to basolateral chambers on Day 0 and Day 1. On Day 2 (48 hrs post virus inoculation), sampling was conducted by adding 200 μL of OptiMEM™ culture media to the apical surface of each well for 10 min (being stored at −80° C.).

Virus load was quantified by RT-PCR as reported by Pizzorno et al. (Cell Rep Med. 1 (4): 100059, 2020). Supernatant will be lysed and viral RNA extracted with the QIAamp Viral RNA Mini Kit (Qiagen). Viral RNA will be quantified by RT-qPCR (Express One-Step Superscript™ qRT-PCR kit, Invitrogen). SARS-COV-2-specific primer and probes used for viral genome quantification as follows: Target: ORF1b-nsp14 Forward primer (HKU-ORF1b-nsp14F) 5′-TGGGGYTTTACRGGTAACCT-3′ Reverse primer (HKU-ORF1b-nsp14R) 5′-AACRCGCTTAACAAAGCACTC-3′ Probe (HKU-ORF1b-nsp141P) 5′-FAM-TAGTTGTGATGCWATCATGACTAG-TAMRA-3′. Ct data were determined and relative changes in gene expression were calculated using the 2-4Ct method and shown as the fold reduction of genome copy number (ORF1b-nsp14 gene of SARS-COV-2) relative to the mean of vehicle treated infected control.

Transepithelial electrical resistance (TEER) was measured to investigate the integrity of tight junction dynamics in air-liquid interface cultured pseudostratified epithelium before and after SARS-COV-2 infection as a surrogate for epithelial damage. Chopstick-electrodes were placed in the apical and basolateral chambers and the TEER was measured using a dedicated volt-ohm meter (EVOM2, Epithelial Volt/Ohm Meter for TEER) and expressed as Ohm/cm².

### Results

High level of SARS-COV-2 replication was detected on apical wash from virus control (RT-PCR: Ct=12.1) at 48 hrs post inoculation. When compared the viral load of each treatment with that in mock control (=1), apilimod alone showed only marginal effects (0.1 log reduction) and surfactants exhibited 1.3 Log reduction of viral load. However, the combination of apilimod and surfactants showed synergistic effects on reducing viral load, leading to 2.2 Log reduction compared with virus infection control (see FIG. 2). Assay control, remdesivir (5 μM) showed 3.4 log reduction of viral load, as expected (Pizzorno et al., 2020, Cell Rep Med.1 (4): 100059).

SARS-COV-2 infection significantly reduced the value of TEER at 48 hrs post virus inoculation in vehicle treated virus infected control. The rapid damage of epithelium by virus was demonstrated by dispersed zonula occludens-1 (ZO-1) expression by immunohistochemistry without clear tight junctions and partial loss of cilia. (Hao et al., 2020, mBio, 11 (6): e02852-20). In contrast, apilimod alone showed partial inhibition of virus-induced reduction in TEER (48% effect). However surfactants and a combination of apilimod and surfactants completely restored the TEER and also further increased the TEER level (see FIG. 3). Thus, these treatments protected from virus-induced cell damage, and further strengthen the epithelial barrier by repairment or cell proliferation. Assay control, remdesivir (5 μM) also protected by 81%, as expected (Pizzorno et al., 2020, Cell Rep Med.1 (4): 100059).

Anti-viral effects were evaluated using air-liquid cultured human primary nasal epithelial cells. The cells undergo extensive mucociliary differentiation, resulting in cultures with morphological characteristics similar to those observed in the normal human nasal epithelium. As a result, this cell model closely mimics SARS-COV-2 infections in human nasal cavity.

### Example 3: Assessment of Virus Load and Cell Integrity in Air-Liquid Interface (ALI) Cultured Human Epithelial Cells Infected with SARS-COV-2-Studies with Oseltamivir Carboxylate

### Experimental Methods

ALI cultured pooled donors' human nasal epithelium (provided by Epithelix Sarl (Geneva, Switzerland)) were maintained in air-liquid interphase with MucilAir™ culture media in Costar Transwell inserts (Corning, NY, USA) according to the manufacturer's instructions. On Day 0, H1N1 inoculum (strain A/Switzerland/7717739/2013 (H1N1), 100 uL; diluted in MucilAir culture medium to give a 4.85E3 genome copy number/ml) was added to apical surface for 1.5 hr (34° C./5% CO2). Virus inoculum was removed, and inserts were washed with sterile media.

ALI cultures were dosed apically with oseltamivir carboxylate (1 μM), surfactants (0.15% polysorbate 80 and 0.2% oleic acid) or oseltamivir carboxylate in surfactants at 10 min prior to virus inoculation, and further 90 min together with virus inoculum on Day 0 (then being removed together with virus inoculum as indicated above), and also re-applied to the apical surface on Day 1 (24 hrs post inoculation) for 10 min before being removed. Vehicle treatments (water) were performed on the corresponding apical surfaces to ensure each well received the same number of manipulations. In addition, oseltamivir carboxylate (10 μM) was added to basolateral chambers on Day 0. On Day 1 and 2 (24 and 48 hrs post virus inoculation), sampling was conducted by adding 200 μL of culture media to the apical surface of each well for 20 min (being stored at −80° C.). Oseltamivir carboxylate is the active metabolite of oseltamivir, the active ingredient of the commercial product Tamiflu®. (oseltamivir phosphate) and is generated in vivo after administration.

Supernatant were lysed and viral RNA extracted with the QIAamp 96 virus QUAcube HT kit (Qiagen). Viral RNA will be quantified by RT-qPCR (QuantiTect Probe RT-PCR, Qiagen) with the qTOWER3 detection system. Ct data were reported to the standard curve and presented as genome copy number/ml. Transepithelial electrical resistance (TEER) was measured as shown above.

### Results

High level of H1N1 replication was detected on apical wash from virus control at 24 hrs post inoculation. When compared the viral load of each treatment with that in mock control, oseltamivir carboxylate alone showed only marginal effects (1.0 log reduction) and surfactants exhibited no reduction of viral load. However, the combination of oseltamivir carboxylate and surfactants showed synergistic effects on reducing viral load, leading 2.2 Log reduction compared with virus infection control (see FIG. 4). Assay control, oseltamivir carboxylate (10 UM in a basal chamber) showed 1.7 log reduction of viral load.

H1N1 infection only slightly reduced the value of trans-epithelial electrical resistance (TEER: as a hallmark of epithelial integrity) on 24 hrs post infection, and strongly reduced it on 48 hrs post infection compared with non-infection control. At 48 hrs post infection, oseltamivir carboxylate alone and surfactants alone respectively fully and partially restored the TEER levels reduced by H1N1, but a combination of oseltamivir carboxylate and surfactants further increased the TEER level (see FIG. 5). Thus, these treatments protected from virus-induced cell damage, and further strengthen the epithelial barrier by repairment or cell proliferation. Assay control, oseltamivir carboxylate (10 μM, basal treatment) also protected against loss of epithelial integrity.

### Summary of Biological Data

The in vitro anti-viral activity of apilimod, surfactants and their combination has been demonstrated by reduction of SARS-COV-2 viral load in infected nasal epithelium. In this assay system the inhibition of virus replication was detected and quantified from the resulting reduction of virus genome by RT-PCR. The surfactants were highly active and there was marked and apparently synergic anti-viral effect of the combination of apilimod and surfactants. The superior anti-viral effect of a combination of oseltamivir and surfactants were also confirmed on influenza virus infected nasal epithelium.

The value of trans-epithelial electrical resistance (TEER: as a hallmark of epithelial integrity) was also found to be significantly reduced after SARS-COV-2 infection or influenza infection in vehicle treated virus infected control. The use of apilimod led to partial inhibition of SARS-CoV-2-induced reduction in in TEER and the use of surfactants and especially a combination of apilimod and surfactants completely inhibited SARS-COV-2-induced reduction in TEER or even increased TEER. Oseltamivir carboxylate alone and surfactants alone respectively fully and partially restored the TEER levels reduced by influenza virus infection, but a combination of oseltamivir carboxylate and surfactants further increased the TEER level. These results suggest that the treatments protect from virus-induced cell damage and also enhance barrier integrity by repairment or cell proliferation. Accordingly, both treatment and prevention effects of the administration are evident.

The results show that liquid pharmaceutical formulations of the invention containing a surfactant component comprising oleic acid as described herein and especially liquid pharmaceutical formulations containing a surfactant component comprising oleic acid as described herein and an anti-viral agent such as apilimod or oseltamivir carboxylate when administered topically to the lung or nose are expected to be useful for the treatment or prevention of viral infections such as SARS-COV-2 and disease associated therewith such as COVID-19 or influenza.

Although the combination of surfactants and the anti-viral agents apilimod and oseltamivir carboxylate was most potent in reducing viral load and restoring TEER, the surfactants alone had marked activity in reducing viral load and restoring TEER during SARS-COV-2 infection. The surfactants alone also had marked activity in restoring TEER during influenza virus infection The effect of restoring TEER (a surrogate for barrier integrity) supports the use of surfactant alone and together with an anti-viral agent to prevent viral infection as well as treat it.

### REFERENCES

Cantuti-Castelvetri L, Ojha R, Pedro L D, Djannatian M, Franz J, Kuivanen S, van der Meer F, Kallio K, Kaya T, Anastasina M, Smura T, Levanov L, Szirovicza L, Tobi A, Kallio-Kokko H, Österlund P, Joensuu M, Meunier F A, Butcher S J, Winkler M S, Mollenhauer B, Helenius A, Gokce O, Teesalu T, Hepojoki J, Vapalahti O, Stadelmann C, Balistreri G, Simons M. Neuropilin-1 facilitates SARS-COV-2 cell entry and infectivity. Science. 2020 Nov. 13; 370 (6518): 856-860. doi: 10.1126/science.abd2985. Epub 2020 Oct. 20. PMID: 33082293; PMCID: PMC7857391.
Cattaneo D, Gervasoni C, Corbellino M, Galli M, Riva A, Clementi E. Does lopinavir really inhibit SARS-COV-2? Pharmacol Res. 2020 August; 158:104898. doi: 10.1016/j.phrs.2020.104898. Epub 2020 May 11. PMID: 32438034; PMCID: PMC7211645.
Chai H, Zhao Y, Zhao C, Gong P. Synthesis and in vitro anti-hepatitis B virus activities of some ethyl 6-bromo-5-hydroxy-1H-indole-3-carboxylates. Bioorg Med Chem. 2006 Feb. 15; 14 (4): 911-7. doi: 10.1016/j.bmc.2005.08.041. Epub 2005 Sep. 23. PMID: 16183290.
Choy K T, Wong A Y, Kaewpreedee P, Sia S F, Chen D, Hui KPY, Chu DKW, Chan MCW, Cheung P P, Huang X, Peiris M, Yen H L. Remdesivir, lopinavir, emetine, and homoharringtonine inhibit SARS-COV-2 replication in vitro. Antiviral Res. 2020 June; 178:104786. doi: 10.1016/j.antiviral.2020.104786. Epub 2020 Apr. 3. PMID: 32251767; PMCID: PMC7127386.
Dragovich P S, Prins T J, Zhou R, et al. Structure-based design, synthesis, and biological evaluation of irreversible human rhinovirus 3C protease inhibitors. 4. Incorporation of P₁ lactam moieties as L-glutamine replacements. J Med Chem. 1999. 42 (7): 1213-1224.
Ford N, Vitoria M, Rangaraj A, Norris S L, Calmy A, Doherty M. Systematic review of the efficacy and safety of antiretroviral drugs against SARS, MERS or COVID-19: initial assessment. J Int AIDS Soc. 2020 April; 23 (4): e25489. doi: 10.1002/jia2.25489. PMID: 32293807; PMCID: PMC7158851
Pizzorno A, Padey B, Julien T, Trouillet-Assant S, Traversier A, Errazuriz-Cerda E, Fouret J, Dubois J, Gaymard A, Lescure F X, Dulière V, Brun P, Constant S, Poissy J, Lina B, Yazdanpanah Y, Terrier O, Rosa-Calatrava M. Characterization and Treatment of SARS-COV-2 in Nasal and Bronchial Human Airway Epithelia. Cell Rep Med. 2020 Jul. 21; 1 (4): 100059. doi: 10.1016/j.xcrm.2020.100059. PMID:
Hao S, Ning K, Kuz C A, Vorhies K, Yan Z, Qiu J. Long-Term Modeling of SARS-COV-2 Infection of In Vitro Cultured Polarized Human Airway Epithelium. mBio. 2020 Nov. 6; 11 (6): e02852-20. doi: 10.1128/mBio.02852-20. PMID: 33158999; PMCID: PMC7649230.
Hoffmann M, Schroeder S, Kleine-Weber H, Müller M A, Drosten C, Pohlmann S. Nafamostat Mesylate Blocks Activation of SARS-COV-2: New Treatment Option for COVID-19. Antimicrob Agents Chemother. 2020 May 21; 64 (6): e00754-20. doi: 10.1128/AAC.00754-20. PMID: 32312781; PMCID: PMC7269515.
Hoffmann M, Mosbauer K, Hofmann-Winkler H, Kaul A, Kleine-Weber H, Krüger N, Gassen N C, Müller M A, Drosten C, Pöhlmann S. Chloroquine does not inhibit infection of human lung cells with SARS-COV-2. Nature. 2020 September; 585 (7826): 588-590. doi: 10.1038/s41586-020-2575-3. Epub 2020 Jul. 22. PMID: 32698190.
Hoffmann M, Hofmann-Winkler H, Smith J C, Krüger N, Arora P, Sørensen L K, Søgaard O S, Hasselstrøm J B, Winkler M, Hempel T, Raich L, Olsson S, Danov O, Jonigk D, Yamazoe T, Yamatsuta K, Mizuno H, Ludwig S, Noe F, Kjolby M, Braun A, Sheltzer J M, Pöhlmann S. Camostat mesylate inhibits SARS-COV-2 activation by TMPRSS2-related proteases and its metabolite GBPA exerts antiviral activity. EBioMedicine. 2021 March; 65:103255. doi: 10.1016/j.ebiom.2021.103255. Epub 2021 Mar. 4. PMID: 33676899; PMCID: PMC7930809.
Huang D, Yu H, Wang T, Yang H, Yao R, Liang Z. Efficacy and safety of umifenovir for coronavirus disease 2019 (COVID-19): A systematic review and meta-analysis. J Med Virol. 2021 January; 93 (1): 481-490. doi: 10.1002/jmv.26256. Epub 2020 Jul. 14. PMID: 32617989; PMCID: PMC7361300.
Kabinger F, Stiller C, Schmitzová J, Dienemann C, Kokic G, Hillen H S, Höbartner C, Cramer P. Mechanism of molnupiravir-induced SARS-COV-2 mutagenesis. Nat Struct Mol Biol. 2021 September; 28 (9): 740-746. doi: 10.1038/s41594-021-00651-0. Epub 2021 Aug. 11. PMID: 34381216; PMCID: PMC8437801
Kadam R U, Wilson I A. Structural basis of influenza virus fusion inhibition by the antiviral drug Arbidol. Proc Natl Acad Sci USA. 2017 Jan. 10; 114 (2): 206-214. doi: 10.1073/pnas. 1617020114. Epub 2016 Dec. 21. PMID: 28003465; PMCID: PMC5240704.
Owen, D. R., Allerton, C., Anderson, A. S., Aschenbrenner, L. M., Avery, M., Berritt, S., Boras, B. W., Cardin, R. D., Carlo, A. A., Coffman, K. J., Dantonio, A., Di, L. S., Eng, H., Ferre, R. A., Gajiwala, K. S., Gibson, S. A., Greasley, S. E., Hurst, B., Kadar, E. P., Kalgutkar, A. S., Lee, J. C., Lee, J., Liu, W., Mason, S. W., Noell, S. E., Novak, J. J., Obach, R. S., Ogilvie, K., Patel, N. C., Pettersson, M., Rai, D. K., Reese, M. R., Sammons, M. F., Sathish, J. G., Singh, R., Steppan, C., Stewart, A. E., Tuttle, J. B., Updyke, L. W., Verhoest, P. R., Wei, L., Yang, Q., & Zhu, Y. (2021). An oral SARS-COV-2 Mpro inhibitor clinical candidate for the treatment of COVID-19. Science, eabl4784.
Marple V A, Roberts D L, Romay F J, Miller N C, Truman K G, Van Oort M, Olsson B, Holroyd M J. Mitchell J P, Hochrainer D. Next generation pharmaceutical impactor (a new impactor for pharmaceutical inhaler testing). Part I: Design. J Aerosol Med. 2003 Fall; 16 (3): 283-99. dor 10.1089/089426803769017659 PMID: 14572326.
Pruijssers A J, George A S, Schäfer A, Leist S R, Gralinksi L E, Dinnon K H 3rd, Yount B L, Agostini M L, Stevens L J, Chappell J D, Lu X, Hughes™, Gully K, Martinez D R, Brown A J, Graham R L, Perry J K, Du Pont V, Pitts J, Ma B, Babusis D, Murakami E, Feng J Y, Bilello J P, Porter D P, Cihlar T, Baric R S, Denison M R, Sheahan T P. Remdesivir Inhibits SARS-COV-2 in Human Lung Cells and Chimeric SARS-COV Expressing the SARS-COV-2 RNA Polymerase in Mice. Cell Rep. 2020 Jul. 21; 32 (3): 107940. doi: 10.1016/j.celrep.2020.107940. Epub 2020 Jul. 7. PMID: 32668216; PMCID: PMC7340027.
Robinot R, Hubert M, Dias de Melo G, Lazarini F, Bruel T, Smith N, Levallois S, Larrous F, Fernandes J, Gellenoncourt S, Rigaud S, Gorgette O, Thouvenot C, Trébeau C, Mallet A, Dumenil G, Gobaa S, Etournay R, Lledo P M, Lecuit M, Bourhy H, Duffy D, Michel V, Schwartz O, Chakrabarti L A. SARS-COV-2 infection damages airway motile cilia and impairs mucociliary clearance. bioRxiv 2020.10.06.328369; doi: https://doi.org/10.1101/2020.10.06.328369
Senokuchi K, Nakai H, Nakayama Y, Odagaki Y, Sakaki K, Kato M, Maruyama T, Miyazaki T, Ito H, Kamiyasu K, et al. New orally active serine protease inhibitors. J Med Chem. 1995 Jul. 7; 38 (14): 2521-3. doi: 10.1021/jm00014a003. PMID: 7629790.
Shirvaliloo M. The blood-gas barrier in COVID-19: an overview of the effects of SARS-COV-2 infection on the alveolar epithelial and endothelial cells of the lung. Tissue Barriers. 2021 Oct. 2; 9 (4): 1937013. doi: 10.1080/21688370.2021.1937013. Epub 2021 Jul. 7. PMID: 34232823.
Unoh Y, Uehara S, Nakahara K, Nobori H, Yamatsu Y, Yamamoto S, Maruyama Y, Taoda Y, Kasamatsu K, Suto T, Kouki K, Nakahashi A, Kawashima S, Sanaki T, Toba S, Uemura K, Mizutare T, Ando S, Sasaki M, Orba Y, Sawa H, Sato A, Sato T, Kato T, Tachibana Y. Discovery of S-217622, a Noncovalent Oral SARS-COV-2 3C L Protease Inhibitor Clinical Candidate for Treating COVID-19. J Med Chem. 2022 May 12; 65 (9): 6499-6512.
Xie L, Zhang Z, Wang Q, Chen Y, Lu D, Wu W. COVID-19 and Diabetes: A Comprehensive Review of Angiotensin Converting Enzyme 2, Mutual Effects and Pharmacotherapy. Front Endocrinol (Lausanne). 2021; 12:772865. Published 2021 Nov. 19. doi: 10.3389/fendo.2021.772865
Zhao Y, Fang C, Zhang Q, Zhang R, Zhao X, Duan Y, Wang H, Zhu Y, Feng L, Zhao J, Shao M, Yang X, Zhang L, Peng C, Yang K, Ma D, Rao Z, Yang H. Crystal structure of SARS-COV-2 main protease in complex with protease inhibitor PF-07321332. Protein Cell. 2021 Oct. 22:1-5. doi: 10.1007/s13238-021-00883-2. Epub ahead of print. PMID: 34687004; PMCID: PMC8533666.

### GENERAL STATEMENTS

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents, patent applications and references mentioned throughout the specification of the present invention are herein incorporated in their entirety by reference.

The invention embraces all combinations of preferred and more preferred groups and suitable and more suitable groups and embodiments of groups recited above.

1. A liquid pharmaceutical formulation suitable for topical administration to the lung or nose comprising (i) a surfactant component selected from the group consisting of oleic acid or a pharmaceutically acceptable salt thereof and mixtures of oleic acid or a pharmaceutically acceptable salt thereof with polyoxyethylene sorbitan fatty acid esters and (ii) an anti-viral agent.

2. The liquid pharmaceutical formulation according to claim 1, wherein the surfactant component is selected from the group consisting of oleic acid or a pharmaceutically acceptable salt thereof and mixtures of oleic acid or a pharmaceutically acceptable salt thereof with polysorbate 80 or polysorbate 20.

3. The liquid pharmaceutical formulation according to claim 2, wherein the surfactant component is a mixture of oleic acid or a pharmaceutically acceptable salt thereof with polysorbate 80.

4. The liquid pharmaceutical formulation according to claim 1, wherein the oleic acid is in the form of the free acid.

5-22. (canceled)

23. The liquid pharmaceutical formulation according to claim 1, wherein the liquid pharmaceutical formulation is an aqueous formulation.

24. (canceled)

25. The liquid pharmaceutical formulation according to claim 1, wherein the liquid pharmaceutical formulation is a pressured liquid formulation.

26-37. (canceled)

38. A method of treating or preventing viral infection or disease associated with infection by a virus or disease associated with infection with such a virus which comprises administering to a subject in need thereof topically to the lung or nose a therapeutically or prophylactically effective amount of a pharmaceutical formulation according to claim 1.

39. The method according to claim 38, wherein the virus is SARS-COV-2 and the disease associated with infection with such a virus is COVID-19; or
wherein the virus is influenza and the disease associated with infection with such a virus is influenza; or
wherein the virus is coronavirus e.g. seasonal coronavirus e.g. 229E and the disease associated with viral infection is the disease associated with coronavirus infection e.g. seasonal coronavirus e.g. 229E infection; or
wherein the virus is respiratory syncytial virus (RSV) and the disease associated with viral infection is the disease associated with RSV infection; or
wherein the virus is human rhinovirus (HRV) and the disease associated with viral infection is the disease associated with HRV infection.

40-49. (canceled)

50. A method of treating or preventing viral infection or disease associated with viral infection which comprises administering to a subject in need thereof topically to the lung or nose a therapeutically or prophylactically effective amount of a pharmaceutical formulation comprising a surfactant component selected from the group consisting of oleic acid and mixtures of oleic acid with polyoxyethylene sorbitan fatty acid esters.

51. The method according to claim 50, wherein the viral infection is SARS-COV-2 infection and the disease associated with viral infection is COVID-19; or
wherein the viral infection is influenza virus infection and the disease associated with viral infection is influenza; or
wherein the viral infection is coronavirus e.g. seasonal coronavirus e.g. 229E infection and the disease associated with viral infection is the disease associated with coronavirus infection e.g. seasonal coronavirus e.g. 229E infection; or
wherein the viral infection is respiratory syncytial virus (RSV) infection and the disease associated with viral infection is the disease associated with RSV infection; or
wherein the viral infection is human rhinovirus (HRV) infection and the disease associated with viral infection is the disease associated with HRV infection.

52-55. (canceled)

56. The method according to claim 50, wherein the liquid pharmaceutical formulation is a pressured liquid formulation.

57. The method according to claim 50, wherein the liquid pharmaceutical formulation is an aqueous formulation.

58. A liquid pharmaceutical formulation suitable for topical administration to the lung or nose comprising a surfactant component selected from the group consisting of oleic acid or a pharmaceutically acceptable salt thereof and mixtures of oleic acid or a pharmaceutically acceptable salt thereof with polyoxyethylene sorbitan fatty acid esters.

59. The liquid pharmaceutical formulation according to claim 58, wherein the surfactant component is selected from the group consisting of oleic acid or a pharmaceutically acceptable salt thereof and mixtures of oleic acid or a pharmaceutically acceptable salt thereof with polysorbate 80 or polysorbate 20.

60. The liquid pharmaceutical formulation according to claim 59, wherein the surfactant component is a mixture of oleic acid or a pharmaceutically acceptable salt thereof with polysorbate 80.

61. The liquid pharmaceutical formulation according to claim 58, wherein the oleic acid is in the form of the free acid.

62. The liquid pharmaceutical formulation according to claim 58, wherein the liquid pharmaceutical formulation is an aqueous formulation.

63. The liquid pharmaceutical formulation according to claim 58, wherein the liquid pharmaceutical formulation is a pressured liquid formulation.

64. The liquid pharmaceutical formulation according to claim 50, wherein the surfactant component is selected from the group consisting of oleic acid or a pharmaceutically acceptable salt thereof and mixtures of oleic acid or a pharmaceutically acceptable salt thereof with polysorbate 80 or polysorbate 20.

65. The method according to claim 64, wherein the surfactant component is a mixture of oleic acid or a pharmaceutically acceptable salt thereof with polysorbate 80.
